(19) United States
(12) Patent Application Publication
(10) Pub. No.: US 2020/0188140 A1
(43) Pub. Date: Jun. 18, 2020

(54) SURGICAL IMPLANT FOR SUPPORTING A PROSTHETIC DEVICE

(71) Applicants: ALFRED HEALTH, Melbourne, Victoria (AU); MONASH UNIVERSITY, Clayton, Victoria (AU)

(72) Inventors: Matthias RUSS, Victoria (AU); Wingkong CHIU, Victoria (AU); Mark FITZGERALD, Victoria (AU)

(21) Appl. No.: 16/623,223

(22) PCT Filed: Jun. 15, 2018
(86) PCT No.: PCT/AU2018/050596

§ 371 Date: Dec. 16, 2019

(30) Foreign Application Priority Data
Jun. 16, 2017 (AU) 2017902308

(51) Int. Cl. A61F 2/78; A61F 2/28; A61B 17/74

(52) U.S. Cl. CPC A61F 2/78; A61F 2/2814; A61F 2002/3093; A61F 2002/7887; A61B 17/744

(57) ABSTRACT

A surgical implant for supporting a prosthetic device at a distal end of an amputated limb, including:

a cup having a base and surrounding side wall for defining therein a volume for accommodating a terminal end of a bone of the amputated limb;

a central intramedullary stem extending from the cup base for insertion into a medullary cavity of the bone;

a plurality of extramedullary struts extending from the side wall generally parallel to the central intramedullary stem and locatable in contact with or in a closely adjacent relation with an outer surface of the bone; and

an attachment member extending from an outer face of the cup base.

### CROSS-REFERENCE TO RELATED APPLICATIONS

This application is a national phase application of International Application No. PCT/AU2018/050596, filed Jun. 15, 2018, which claims priority to Australian Patent Application Ser. No. 2017902308, filed Jun. 16, 2017, all of which are incorporated herein by reference.

### BACKGROUND

The present invention is generally directed to prosthetic devices, and in particular to a surgical implant for supporting a prosthetic device.

Following amputation of a leg or arm, at least a part of the function of the amputated limb can be achieved using a prosthetic device. It is necessary to provide means to securely attach the prosthetic device to the distal end of the amputated limb. Surgical implants having means to allow the prosthetic device to be mounted on the amputated limb have been proposed.

In U.S. Pat. No. 7,374,577 B2 (Shin Ki Kim et al.), there is described an implant device that can be mounted to an amputated end of a thighbone or lower leg bone by insertion of an implant insert into the centre of the bone. It is therefore necessary to ream the bone before the implant insert can be inserted. Other components of the implant device can be secured to the implant insert by a coupling screw. Loads applied to the implant by an attached prosthesis are therefore primarily carried by the implant insert.

In U.S. Pat. No. 7,909,883 B2 (Christopher G. Sidebotham), there is described an implant device using a tapered internal stem. The bone must be reamed before the tapered internal stem can be inserted into the bone. That internal stem must then stabilise and carry the load applied by the prosthesis secured to the implant device. A porous outer body member is also provided around part of the internal stem to encourage bone integration.

In US 20140228896 A1 (Harry N. Smith), there is described a shock absorbing implant device that is secured to the terminal end of the bone of an amputated limb by means of an internal screw mechanism. This device requires the terminal end to be pre-tapped to fit the screw device, or to have a thread housing cemented in place. All loads, including lateral loads, applied to the device is carried by the internal screw mechanism.

Each of the above described implant devices have a number of disadvantages. Firstly, each device requires the bone to be reamed or pre-tapped prior to the installation of the device. This generates excessive heat that can cause damage to the surrounding blood vessels limiting the speed of osseointegration between the amputated limb and the implant and thereby increasing the recovery time of the amputee. Furthermore, as the load applied by the prosthesis is primarily carried by the implant device, this can lead to bone atrophy as a result of stress shielding due to the removal of the loads normally carried by the bone by the implant device. In addition, loads cannot be immediately applied to the implant device because time is required to allow for osseointegration between the device and the surrounding bone tissue, or the setting of cement used to secure the implant. This period of unloading of the implant can also lead to bone atrophy.

It is therefore an object of the present invention to provide a surgical implant for supporting a prosthetic device that overcomes one or more of the disadvantages associated with known implant devices.

The above discussion of background art is included to explain the context of the present invention. It is not to be taken as an admission that any of the documents or other material referred to was published, known or part of the common general knowledge at the priority date of any one of the claims of this specification.

### SUMMARY

According to one aspect of the present invention, there is provided a surgical implant for supporting a prosthetic device at a distal end of an amputated limb, including:

a cup having a base and surrounding side wall for defining therein a volume for accommodating a terminal end of a bone of the amputated limb;

a central intramedullary stem extending from the cup base for insertion into a medullary cavity of the bone;

a plurality of extramedullary struts extending from the side wall generally parallel to the central intramedullary stem and locatable in contact with or in a closely adjacent relation with an outer surface of the bone; and

an attachment member extending from an outer face of the cup base.

The dimension and shape of the intramedullary stem may be selected to minimise stress shielding of the bone. According to one possible embodiment of the invention, the intramedullary stem may be tapered. The intramedullary stem may be joined to the cup base through a generally frustoconical section. This acts to compact the marrow within the bone which can lead to initial stability of the implant when installed and can help to achieve osseointegration of the implant once installed.

The dimension and shape of each extramedullary strut may be selected to stabilise the implant when fitted, and to minimise stress shielding of the bone. According to one possible embodiment of the invention, each extramedullary strut may have a curved profile closely corresponding to the bone outer surface. This facilitates the extramedullary struts being in contact or closely adjacent to the bone outer surface. Each extramedullary strut may have a peripheral edge that tapers from the cup surface, preferably to a rounded lip.

The extramedullary struts act to stabilise the implant once fitted. Furthermore, the shape of the extramedullary struts allows the bone tissue to carry at least part of the load applied to the implant by an attached prosthetic device, thereby minimising stress shielding as previously described.

The attached member may be a prosthesis stem extending from the outer face of the cup base. This stem can be suitably configured to facilitate mounting of prosthetic device thereon.

The implant may have a coating agent that can facilitate osseointegration between the implant and the bone. That coating agent may preferably be hydroxyapatite. It is however to be appreciated that the use of other coating agents that can achieve this objective is also envisaged.

Olive wires may pass through and extend between opposing extramedullary struts when the implant is fitted.

The surgical implant may further include a surface coating on an outer surface of the implant to encourage skin growth of the amputated limb into the outer surface surrounding the attachment member.

According to another aspect of the present invention, there is provided a method of surgically fitting an implant as described above including:

press fitting the implant to the terminal end of a bone of an amputated limb by inserting and pressing the intramedullary stem into the medullary cavity of the bone such that the terminal end of the bone is accommodated within the cup, and the extramedullary struts extend in contact with or in a closely adjacent relation with the outer surface of the bone.

The method may further include passing olive wires through and between opposing extramedullary struts and through the bone to reinforce the extramedullary struts.

The method may further include thinning a soft tissue flap of the amputated limb surrounding the attachment member of the implant when fitted, and pressing said soft tissue flap against the outer surface of the implant surrounding the attachment member to encourage skin growth into said outer surface.

The surgical implant according to the present invention can be press fitted to the terminal end of the bone eliminating the need for any reaming or pre-tapping of bone prior to installation. Potential damage to the bone or surrounding blood vessels can be avoided. Also, the implant can be used straight away because it is press-fitted, and not connected to the bone which requires a period of time before the insert can be used. Furthermore, the provision of the extramedullary struts to distribute more of the load to the surrounding bone, as well as being able to immediately load the implant, both act to limit thereby limit stress shielding of the bone. The press fitting of the implant also makes it easier to replace in future. The implant according to the present invention may be relatively smaller in size than known implants. This can therefore allow for installation of this implant close to other device such as a hip replacement.

### BRIEF DESCRIPTION OF THE DRAWINGS

It will be convenient to further describe the invention with respect to the accompanying drawings which illustrate a preferred embodiment of the surgical implant of the present invention. Other embodiments of the invention are possible, and consequently, the particularity of the accompanying drawings is not to be understood as superseding the generality of the preceding description of the invention.

In the drawings:

FIG. 1 is a perspective view of a surgical implant for supporting a prosthetic device at a distal end of an amputated limb according to the present invention;

FIG. 2 is a cross-sectional view of the surgical implant of FIG. 1; and

FIG. 3 is a photographic image of a bone model upon which has been fitted with the surgical implant of FIG. 1.

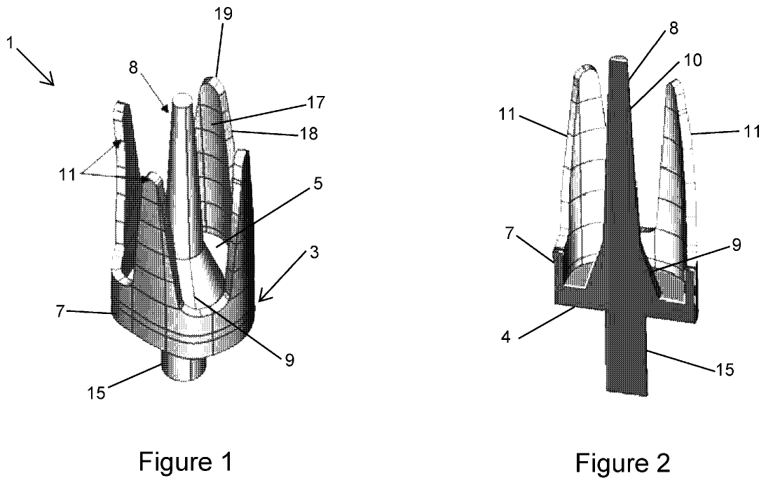

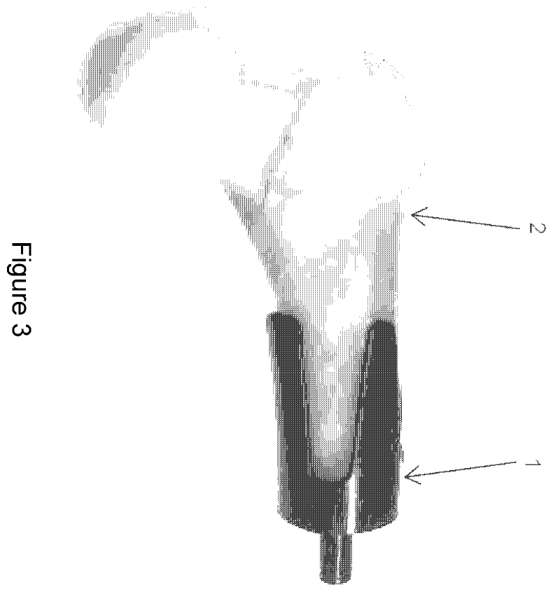

### DETAILED DESCRIPTION

The surgical implant according to the present invention needs to be custom made to suit the amputee. This therefore requires the partially amputated limb to be imaged using imaging equipment such as a CT scan or an MRI scan. Once a 3D scan of the amputated limb has been generated, the surgical implant can be modified to conform to the specific geometry of the amputated limb.

The surgical implant 1 includes a cup 3 having a base 4 and a surrounding side wall 7. The cup 3 defines a volume 5 adapted to accommodate a terminal end of a bone 2 of the amputated limb.

Extending from the cup base 4 and through the accommodating volume 5 is a central intramedullary stem 8. The intramedullary stem 8 includes a frustoconical base portion 9 extending from the base 4 and a tapered upper portion 10. The accommodating volume 5 is shaped to closely accommodate the terminal end of the bone 2. The tapered shape of the stem 8 helps to minimise shielding of the loading applied to the bone that can lead to bone atrophy. Also, the frustoconical base portion 9 can act to compact the cancellous bone (i.e. marrow), helping to get better initial stability of the implant 1 when first fitted. This also helps to achieve osseointegration of the implant 1 to the bone 2.

Extending from the cup side wall 7 are a plurality of extramedullary struts 11. While four struts 11 are shown in the drawings, it is to be appreciated that the invention is not limited to this number. The extramedullary struts 11 extend generally parallel to the intramedullary stem 8. When the implant 1 is fully press-fitted onto the bone 2, the extramedullary struts 11 will be in contact with or in a closely adjacent relationship with an outer surface of the bone 2. Each extramedullary strut 11 therefore has a curved inner profile 17 to facilitate the in contact or closely adjacent relation with the bone 2. Each extramedullary strut 11 may have a peripheral edge 18 that tapers from the side wall 7 to a rounded tip 19.

It is envisaged that the extramedullary struts 11 be reinforced using olive wires (not shown). An olive wire has small beads called ‘olives’ therealong. Olive wires may pass through and between the extramedullary struts 11 and through the bone 2 encompassed by the struts 11. Tension on the olive wire will pull the olive and the struts 11 against the bone 2. The olive wires, once inserted and tensioned in opposite directions can produce compression on the bone 2.

On the opposing side of the cup 3 is provided an engagement member in the form of a prosthesis stem 15. A prosthetic device can then be attached to the prosthesis stem 15 using known methods.

The dimensions and shape of the central intramedullary stem 8 and the extramedullary struts 11 may vary depending on the configuration of the bone 2 at its terminal end. This will vary due to the type of limb that has been amputated, and the location of the amputation along the bone. The geometry of the intramedullary stem 8 and the extramedullary struts 11 may therefore be determined to suit the location of the bone terminal end, and to reduce the stress shielding of the bone 2. This may be achieved by ensuring that the intramedullary stem 8 is located away from the walls of the medullary cavity so that the bone 2 is not shielded from loading leading to stress shielding. Otherwise, bone atrophy can occur due to the load carried by the bone being reduced or eliminated.

The dimension and shape of each extramedullary strut 11 are also selected to ensure stability of the implant 1, once fitted. This will depend on factors including the length of the bone and its geometry at the terminal end thereof. As well as providing sufficient strength for the implant 1, the struts 11 are also shaped to minimise stress shielding of the bone 2.

The implant 1 can be produced using a variety of different materials and may use manufacturing processes such as 3D printing or CNC machining depending on the material being used. The implant 1 may be coated with an agent to facilitate osseointegration between the implant 1 and the bone 2. The implant 1 can for example be coated with hydroxyapatite. The use of other coating agents is however also envisaged.

The present invention also proposes a means to prevent ongoing skin irritation and low grade infection through a foreign body reaction on the skin implant interface. Current implants use a concept where the implant has a polished surface which sticks through the soft issues and skin. This creates an ongoing open wound which increases temperature and a skin irritation and possible infection which needs day to day wound care and cleaning to avoid infection. This contributes to a lot of those devices failing and getting loose.

The present invention proposes to thin out the soft tissue flap where the implant leaves the skin and to use a surface coating on an outer surface of the implant 1, in particular surround the prosthesis stem 15, to allow the skin to grow into the implant surface and allow wound healing without creating an open wound. It creates an interface similar to that around an antler or horns in a cow.

It is to be appreciated that the implant 1, once press fitted, can be used immediately. This is because the extramedullary struts 11 act to stabilise the implant 1 once installed. It is not necessary to wait for osseointegration of the implant 1 with the bone 2 before use.

The extramedullary struts 11 according to the present invention therefore facilitates a simple press fit installation of the implant 1, while at the same time limiting the amount of stress shielding exhibited by the bone 2. The extramedullary struts 11, in combination with the central intramedullary stem 8, results in a smaller device which allows for instant loading and limits the amount of stress shielding that occurs.

Modifications and variations that would be deemed obvious to the person skilled in the art are included within the ambit of the present invention as claimed in the appended claims.

1. A surgical implant for supporting a prosthetic device at a distal end of an amputated limb, including:
a cup having a base and surrounding side wall for defining therein a volume for accommodating a terminal end of a bone of the amputated limb;
a central intramedullary stem extending from the cup base for insertion into a medullary cavity of the bone;
a plurality of extramedullary struts extending from the side wall generally parallel to the central intramedullary stem and locatable in contact with or in a closely adjacent relation with an outer surface of the bone; and
an attachment member extending from an outer face of the cup base.

2. A surgical implant according to claim 1, wherein the dimension and shape of the intramedullary stem is selected to minimise stress shielding of the bone.

3. A surgical implant according to claim 1, wherein the intramedullary stem is tapered.

4. A surgical implant according to claim 1, wherein the intramedullary stem is joined to the cup base through a generally frustoconical section.

5. A surgical implant according to claim 1, wherein the dimension and shape of each extramedullary strut is selected to stabilise the implant when fitted and to minimise stress shielding of the bone.

6. A surgical implant according to any claim 1, wherein each extramedullary strut has a curved profile closely corresponding to the bone outer surface.

7. A surgical implant according to claim 6, wherein each extramedullary strut has a peripheral edge that tapers from the cup wall.

8. A surgical implant according to claim 7, wherein each extramedullary strut tapers to a rounded lip thereof.

9. A surgical implant according to claim 1, wherein the attachment member is a prosthesis stem.

10. A surgical implant according to claim 1, further including a coating agent to facilitate osseointegration between the implant and the bone.

11. A surgical implant according to claim 10, wherein the coating agent is hydroxyapatite.

12. A surgical implant according to claim 1, further including olive wires passing through and extending between opposing extramedullary struts when the implant is fitted.

13. A surgical implant according to claim 1, including a surface coating on an outer surface of the implant to encourage skin growth of the amputated limb into the outer surface surrounding the attachment member.

14. A method of surgically fitting a surgical implant to an amputated limb according to claim 1, including:
press fitting the implant to the terminal end of the bone of the amputated limb by inserting the intramedullary stem into the medullary cavity of the bone such that the terminal end of the bone is accommodated within the cup, and the extramedullary struts extend in contact with or in a closely adjacent relation with the outer surface of the bone.

15. A method according to claim 14, including passing olive wires through and between opposing extramedullary struts and through the bone to reinforce the extramedullary struts.

16. A method according to claim 14 when appended to claim 13, further including thinning a soft tissue flap of the amputated limb surrounding the attachment member of the implant when fitted, and pressing said soft tissue flap against the outer surface of the implant surrounding the attachment member to encourage skin growth into said outer surface.
